(19) United States
(12) Patent Application Publication
(10) Pub. No.: US 2018/0103917 A1
(43) Pub. Date: Apr. 19, 2018

(54) HEAD-MOUNTED DISPLAY EEG DEVICE

(71) Applicant: NGOGGLE, San Diego, CA (US)

(72) Inventors: Stanley Kim, San Diego, CA (US); Yuan-Pin Lin, San Diego, CA (US); John Zao, San Diego, CA (US); Felipe Medeiros, San Diego, CA (US); Tzyy-Ping Jung, San Diego, CA (US)

(21) Appl. No.: 15/572,482

(22) PCT Filed: May 8, 2016
(86) PCT No.: PCT/US16/31394

§ 371 Date: Nov. 7, 2017

Related U.S. Application Data
(60) Provisional application No. 62/304,198, filed on Mar. 5, 2016.
(60) Provisional application No. 62/159,190, filed on May 8, 2015.

(51) Int. Cl. A61B 5/00; A61B 5/0478; A61B 5/0484; A61B 5/0496; G02B 27/01

(52) U.S. Cl. CPC A61B 5/7445; A61B 5/0478; A61B 5/04842; A61B 5/7264; A61B 5/6803; G02B 27/017; A61B 5/0496

(57) ABSTRACT

Methods, systems, and devices are disclosed for monitoring electrical signals of the brain. In one aspect, a system for monitoring electrical brain activity associated with visual field of a user includes a sensor unit to acquire electroencephalogram (EEG) signals including a plurality of EEG sensors circumnavigating the head of a user, and a head-mounted frame for docking a personal electronic device over the user's eyes to present visual stimuli, in which the visual stimuli is configured to evoke EEG signals exhibited by the user, in which the assessment indicates if there is a presence of visual field defects in the user's visual field.

### TECHNICAL FIELD

This patent document relates to systems, devices, and processes that use brain machine interface (BMI) technologies.

### BACKGROUND OF THE INVENTION

Diagnosis and detection of progression of neurological disorders remain challenging tasks. For example, a validated portable objective method for assessment of degenerative diseases would have numerous advantages compared to currently existing methods to assess functional loss in the disease. An objective EEG-based test would remove the subjectivity and decision-making involved when performing perimetry, potentially improving reliability of the test. A portable and objective test could be done quickly at home under unconstrained situations, decreasing the required number of office visits and the economic burden of the disease. In addition, a much larger number of tests could be obtained over time. This would greatly enhance the ability of separating true deterioration from measurement variability, potentially allowing more accurate and earlier detection of progression. In addition, more precise estimates of rates of progression could be obtained. The exemplary visual field assessment methods can be used for screening in remote locations or for monitoring patients with the disease in underserved areas, as well as for use in the assessment of visual field deficits in other conditions.

An event-related potential (ERP) is the measured brain response that is the direct result of a specific sensory, cognitive, or motor event. More formally, it is any stereotyped electrophysiological response to a stimulus, and includes event-related spectral changes, event-related network dynamics, and the like. As used herein, the term “visual-event-related potential” (VERP) (also known herein as visual event-related response (VERR) and visually event related cortical potential (VERCP)) refers to a electrophysiological brain response directly or indirectly attributed to a visual stimulation, for example, an indirect brain response as a result of a sensory, cognitive or motor event initiated due to a visual stimulation. Steady-state visual-event-related potentials (SSVERPs) have been shown to be useful for many paradigms in cognitive (visual attention, binocular rivalry, working memory, and brain rhythms) and clinical neuroscience (aging, neurodegenerative disorders, schizophrenia, ophthalmic pathologies, migraine, autism, depression, anxiety, PTSD, stress, and epilepsy) (Vialatte F B, Maurice M, Dauwels J, Cichocki A. Steady-state visually evoked potentials: focus on essential paradigms and future perspectives. Prog Neurobiol. 2010. 90(4):418-38).

Numerous systems and methods are known for examining states of health of eyes. For example, U.S. Pat. No. 5,065,767, issued Nov. 19, 1991 to Maddess, discloses a psychophysical method for diagnosing glaucoma that employs a time varying contrast pattern. Glaucoma may be indicated for an individual who displays a higher than normal contrast threshold for observing the pattern. Maddess also discloses other tests for glaucoma such as the well-known observation of a scotoma, measurement of intraocular pressure, and assessment of color vision defects. U.S. Pat. No. 5,295,495, issued Mar. 24, 1994 to Maddess, discloses systems and methods for diagnosing glaucoma using an individual's response to horizontally moving stripe patterns, which is known as optokinetic nystagmus (OKN). The spatially varying patterns may also vary temporally. In U.S. Pat. No. 5,539,482, issued Jul. 23, 1996 to James et al., additional systems and methods for diagnosing glaucoma using spatial as well as temporal variations in contrast patterns are disclosed. U.S. Pat. No. 5,912,723, issued Jun. 15, 1999 to Maddess, discloses systems and methods that use a plurality of spatially and temporally varying contrast patterns to improve the methods disclosed in the earlier patents. U.S. Pat. No. 6,315,414, issued Nov. 13, 2001 to Maddess et al., describes systems and methods for making a binocular assessment of possible damage to the optical nerve, optical radiations and white matter of the visual brain indicative of various neurological disorders by measuring responses to visual stimuli.

U.S. Pat. No. 6,068,377, issued May 30, 2000 to McKinnon et al., describes systems and methods for testing for glaucoma using a frequency doubling phenomenon produced by isoluminent color visual stimuli. The disclosure is similar to that of Maddess and co-workers, but uses different, preferably complementary, frequencies of light having the same luminosity as the visual probe signal.

U.S. Pat. Nos. 5,713,353 and 6,113,537 describe systems and methods for testing for blood glucose level using light patterns that vary in intensity, color, rate of flicker, spatial contrast, detail content and or speed. The approach described involves measuring the response of a person to one or more light pattern variations and deducing a blood glucose level by comparing the data to calibration data.

Other disease conditions and their identification are described in a paper by S. Sokol, entitled “The visually evoked cortical potential in the optic nerve and visual pathway disorders,” which was published in Electrophysiological testing in diseases of the retina, optic nerve, and visual pathway, edited by G. A. Fishman, published by the American Academy of Opthalmology, of San Francisco, in 1990, Volume 2, Pages 105-141. An article by Clark Tsai, entitled “Optic Nerve Head and Nerve Fiber Layer in Alzheimer's Disease,” which was published in Arch. of Opthalmology, Vol. 107, February, 1991, states that large diameter neurons are damaged in Alzheimer's disease. A review of such tests visual spatial dysfunction associated with a number of neurodegenerative diseases including Alzheimer's disease, Parkinson's disease, Lewy Body Dementias, Corticobasal Syndrome, Progressive Supranuclear Palsy, and Frontotemporal Lobar Degeneration are described in Possin K. L. Visual Spatial Cognition in Neurodegenerative Disease. Neurocase. 2010 December; 16(6): 466-487 (incorporated herein by reference)

U.S. Pat. No. 5,474,081, issued Dec. 12, 1995 to Livingstone et al., describes systems and methods for determining magnocellular defect and dyslexia by presenting temporally and spatially varying patterns, and detecting visual-event-related responses (VERR) using an electrode assembly in contact with the subject being tested.

U.S. Pat. No. 6,129,682, issued Oct. 10, 2000 to Borchert et al., discloses systems and methods for non-invasively measuring intracranial pressure from measurements of an eye, using an imaging scan of the retina of an eye and a measurement of intraocular pressure. The intraocular pressure is measured by standard ocular tonometry, which is a procedure that generally involves contact with the eye. U.S. Pat. Nos. 5,830,139, 6,120,460, 6,123,668, 6,123,943, 6,312,393 and 6,423,001 describe various systems and methods that involve mechanical contact with an eye in order to perform various tests. Direct physical contact with an eye involves potential discomfort and risk of injury through inadvertent application of force or transfer of harmful chemical or biological material to the eye. Direct physical contact with an eye is also potentially threatening to some patients, especially those who are young or who may not fully understand the test that is being performed.

There are few if any currently available reliable and effective portable methods for assessment of functional loss in such disorders.

In addition to being unwieldy, the coupled system often utilizes redundant features, which are not necessary when using the devices together. By way of example, each device utilizes a display screen, which adds cost, size, weight, and complexity to the entire system.

Accordingly, there is a need for a head-mounted EEG display system, particularly a system that temporarily integrates or merges both mechanically and electronically a head-mounted EEG device with a portable electronic device.

### SUMMARY OF THE INVENTION

In accordance with one embodiment of the invention, there is provided a head-mounted neuro-monitoring system and device that is worn on a user's head for visual-field examination by using high-density EEG to associate the dynamics of visual-event-related potentials (VERPs).

In one aspect, there is provided an integrated system and methods for monitoring electrical brain activity of a user that includes 1) a sensor unit to acquire electroencephalogram (EEG) signals from one or more EEG sensors arranged to acquire EEG signals from the head of a user and 2) a portable electronic device (PED) frame to house a removable portable electronic device (PED) with a visual display unit (aka portable visual display) that is temporarily attachable to the head of the user in front of the user's eyes to present visual stimuli. The visual stimuli is configured to evoke visual-event related potentials (VERPs) in the EEG activity signals exhibited by the user and acquired by the sensor unit. The integrated system may further include a data processing unit to process multiple EEG signals and communicate with the sensor unit and the portable electronic device. The processes to analyze the acquired EEG signals and produce an assessment of the user's visual field, in which the assessment indicates if there is a presence of visual dysfunction in the user, may be performed on the data processing unit or utilize the processing unit of the portable electronic device.

In accordance with the invention, a head-mounted EEG system and method of operation are provided in which the system can allow users to physically and/or operatively couple and decouple a portable electronic device with the head-mounted EEG device. The head-mounted EEG device may include a PED frame that is configured to physically receive and carry a portable electronic device. The PED frame may place a display screen of the portable electronic device in front of the user's eyes. The display screen of the portable electronic device may act as the primary display screen of the head-mounted EEG device such that the display screen of the portable electronic device is primarily used to view image-based content when the head-mounted display EEG device is worn on the user's head.

In accordance with yet another embodiment of the invention, there is provided a method for displaying visual stimuli on a head-mounted EEG device. The method may include coupling a portable electronic device to the head-mounted EEG device such that a screen of the portable electronic device faces a user and displays visual stimuli, evoking a brain signal that is monitored using the device. The method may also include providing an instruction to play back visual stimuli stored on or transmitted to the portable electronic device.

The subject matter described in this patent document can be implemented in specific ways that provide one or more of the following features. For example, the disclosed portable platform can facilitate detection, monitoring and assessment of vision dysfunction or impairment such as functional, localized and/or peripheral visual field loss, vision acuity or vision mistakes, or more generally, neural dysfunction. The disclosed portable platform uses high-density EEG recording and visual-event-related responses that can provide improved signal-to-noise ratios, increasing reproducibility and diagnostic accuracy, e.g., as EEG-based methods for objective perimetry such as SSVERP. As a portable platform that could be used for testing in unconstrained situations, the disclosed methods can allow for much broader and more frequent testing of patients, e.g., as compared to existing approaches. For example, this could reduce the number of office visits necessary for patients at risk or diagnosed with optic, neuro-optic or neuro-degenerative disorders. In addition, by allowing more frequent testing, the disclosed methods can facilitate the discrimination of true deterioration from test-retest variability, e.g., resulting in earlier diagnosis and detection of progression and also enhance understanding of how the disease affects the visual pathways. The disclosed portably-implemented and objective methods for visual field assessment can also allow screening for visual loss in underserved populations.

The disclosed technology includes a portable platform that integrates a wearable EEG dry system and a head-mounted EEG display system that allows users to routinely and continuously monitor the electrical brain activity associated with visual field in their living environments, e.g., representing a transformative way of monitoring disease progression. In addition, such devices provide an innovative and potentially useful way of screening for the disease. The disclosed technology includes portable brain-computer interfaces and methods for sophisticated analysis of EEG data, e.g., including capabilities for diagnosis and detection of disease progression.

### BRIEF DESCRIPTION OF THE DRAWINGS

The above and other features of the present invention, its nature and various advantages will be more apparent upon consideration of the following detailed description, taken in conjunction with the accompanying drawings in which:

FIG. 1 shows a simplified diagram of a head-mounted EEG display system in accordance with embodiments of the invention;

FIG. 2 shows a schematic diagram of a portable electronic device docked in docking member in accordance with embodiments of the invention;

FIG. 3 shows perspective views of a head-mounted display EEG device in accordance with embodiments of the invention;

FIG. 4 shows a configuration for sliding a portable electronic device into an alternative configuration of a head-mounted display EEG device in accordance with embodiments of the invention;

FIG. 5 shows a perspective view of a head-mounted EEG display system detecting the user's head movements when mounted on a user's head in accordance with embodiments of the invention;

FIG. 6 shows a flowchart of an illustrative process for displaying image-based content on a portable electronic device in accordance with embodiments of the invention;

FIG. 7 depicts a flowchart for learning;

FIG. 8 shows a flowchart of an illustrative process for comparing EEG signals over time in accordance with embodiments of the invention.

### DETAILED DESCRIPTION OF THE INVENTION

The present invention refers to the field of visual-event-related responses (VERPs), which has been shown to be useful for many paradigms in cognitive (visual attention, binocular rivalry, working memory, and brain rhythms) and clinical neuroscience (aging, neurodegenerative disorders, schizophrenia, ophthalmic pathologies, migraine, autism, depression, anxiety, stress, and epilepsy), particularly to VERP generated by optical stimuli. Accordingly, in one embodiment, the present invention relates to the field of ophthalmologic diagnosis of neurological complications: in particular that of major ocular pathologies like glaucoma, retinal anomalies and of sight, retinal degeneration of the retinal structure and macular degeneration, diabetic retinopathy, amblyopia, optic neuritis, optical neuroma; or degenerative diseases such as Parkinson's disease, Alzheimer's disease, non-Alzheimer's dementia, multiple sclerosis, ALS, head trauma, diabetes, or other cognitive disorders such as dyslexia; or other mental disorders such as obsessive-compulsive disorders. In one particular embodiment, the present invention refers to inappropriate responses to contrast sensitivity patterns, and disorders affecting the optical nerve and the visual cortex.

Optic degeneration, particularly optic neuropathies, can result in significant and irreversible loss of visual function and disability. For example, glaucoma is associated with a progressive degeneration of retinal ganglion cells (RGCs) and their axons, resulting in a characteristic appearance of the optic disc and a concomitant pattern of visual field loss. Loss of visual function in glaucoma is generally irreversible, and without adequate treatment the disease can progress to disability and blindness. The disease can remain relatively asymptomatic until late stages and, therefore, early detection and monitoring of functional damage is paramount to prevent functional impairment and blindness.

It is estimated that glaucoma affects more than 70 million individuals worldwide with approximately 10% being bilaterally blind, which makes it the leading cause of irreversible blindness in the world. However, as the disease can remain asymptomatic until it is severe, the number of affected individuals is likely to be much larger than the number known to have it. Population-level survey data indicate that only 10% to 50% of the individuals are aware they have glaucoma.

Visual dysfunction appears to be a strong predictor of cognitive dysfunction in subject in a number of clinical neuroscience disorders. For example, the functional deficits of glaucoma and Alzheimer's Disease include loss in low spatial frequency ranges in contrast sensitivity, and are similar in both diseases. Pattern masking has been found to be a good predictor of cognitive performance in numerous standard cognitive tests. The tests found to correlate with pattern masking included Gollin, Stroop-Work, WAIS-PA, Stroop-Color, Geo-Complex Copy, Stroop-Mixed and RCPM. Losses in contrast sensitivity at the lowest spatial frequency also was predictive of cognitive losses in the seven tests. AD subjects have abnormal word reading thresholds corresponding to their severity of cognitive impairment and reduced contrast sensitivity in all spatial frequencies as compared to normal subjects.

Similarly, the invention can be used for multiple sclerosis (MS). It is known that MS affects neurons and that the effect comes and goes with time. There is apparent recovery of the cells at least in early stages of the disease. One would therefore expect the diagnosed areas of loss in the visual field to move around the visual field over time, and perhaps to recovery temporarily. As the disease progresses to the point where there is a lot of loss on the retina, the areas of loss will remain lost and will not show temporary recovery.

The retina and brain do parallel processing to determine relative position of adjacent objects. In the case of dyslexia, this processing somehow gets reversed and the subject mixes up the order of letters in words or even the order of entire words. This too could show up as an apparent ganglion cell loss. Again, the apparent loss could be from the ganglion cells or from the feedback to the lateral geniculate nucleus.

Accordingly, the present invention provides an improved apparatus for screening for many optic neuropathy and neuro-degenerative diseases, including Alzheimer's, non-Alzheimer's dementia such as functional dementia, Parkinson's, Schizophrenia multiple sclerosis, macular degeneration, glaucoma, ALS, diabetes, dyslexia, head trauma (such as traumatic brain injury and blast injury), seizures and sub-clinical seizure activity, and possibly others. In one embodiment, the invention can be used to detect onset, or early detection, for example in children, disruptive behavior disorders such as conduct disorder and bipolar disorder, autistic spectrum and pervasive developmental delay, cerebral palsy, acquired brain injury such as concussions, birth trauma, sleep problems that can be helped such as bed wetting, sleep walking, sleep talking, teeth grinding, nightmares, night terrors, adolescence issues including drug abuse, suicidal behavior, anxiety and depression, and in older people for brain function, and other episodic events such as pain, addiction, aggression, anxiety, depression, epilepsy, headaches, insomnia, Tourette syndrome, and brain damage from physical trauma (traumatic brain injury, stroke, aneurysm, surgery, other neurological disorder), illnesses, and injuries, and other causes.

The invention may be further be used for business and marketing applications, based on a person's psychological type/traits, cognitive skill levels, and associated psychological profile for a selected individual or group of individuals; which may include: advertising and marketing, communication skills and team dynamics, consumer behavior, dating service compatibility, human-computer interaction, job placement, leadership and management, organizational development, political messaging, sales, skills development, social networking behavior, as well as media design for books, electronic pads or computer applications, film and television, magazines, questionnaires, and smart phones.

The invention may be used for educational and learning applications, based on a person's psychological type/traits, cognitive skill levels, and any associated psychological profile, for a selected individual or group of individuals; wherein these may include: academic counseling, career counseling, media design for textbooks and electronic pad or computer applications, types of learners and learning modes such as sensory modalities (auditory, tactile, or visual), types of instructors and instructional methods and materials, academic strengths and weaknesses such as concrete verses abstract math learners, the arts, memory retention, mental acuity, training, and the like. The invention can enhance the learning of information, for example, enable the system to customize lessons to individuals and their personalities. Alternatively, the invention may be used for entertainment purposes such as for video games, virtual reality or augmented reality.

A visual-event-related response or evoked response is an electrical potential recorded from the nervous system of a human or other animal following presentation of a visual stimulus. Visually stimulation include patterned and unpatterned stimulus, which include diffuse-light flash, checkerboard and grating patterns, transient VERP, steady-state VERP. flash VERPs, images, games, videos, animation and the like. Some specific VERPs include monocular pattern reversal, sweep visual evoked potential, binocular visual evoked potential, chromatic visual evoked potential, hemi-field visual evoked potential, flash visual evoked potential, LED Goggle visual evoked potential, motion visual evoked potential, multifocal visual evoked potential, multi-channel visual evoked potential, multi-frequency visual evoked potential, stereo-elicited visual evoked potential, steady state visual-event-related response and the like.

Steady state visual-event-related responses (SSVERP), which include steady state visual evoked potentials, are signals that are natural responses to visual stimulation at specific frequencies. When the retina is excited by a visual stimulus ranging from 3.5 Hz to 75 Hz, the brain generates electrical activity at the same (or multiples of) frequency of the visual stimulus. mfSSVERP is a subset of steady-state visual-event-related responses which reflect a frequency-tagged oscillatory EEG activity modulated by the frequency of periodic visual simulation higher than 6 Hz. mfSSVERP is a signal of multi-frequency tagged SSVERP, e.g., which can be elicited by simultaneously presenting multiple continuous, repetitive black/white reversing visual patches flickering at different frequencies. Based on the nature of mfSSVERP, a flicker sector(s) corresponding to a visual field deficit(s) will be less perceivable or unperceivable and thereby will elicit a weaker SSVERP, e.g., as compared to the brain responses to other visual stimuli presented at normal visual spots.

This invention generally pertains to head-mounted electroencephalogram (EEG)-based systems, methods, and devices for visual-field examination by using EEG to associate the dynamics of visual-event-related responses (VERPs) with visual field defects or changes. In one aspect, there is provided an integrated system and methods for monitoring electrical brain activity associated with visual field of a user that includes 1) a sensor unit to acquire electroencephalogram (EEG) signals from one or more EEG sensors arranged to acquire EEG signals from the head of a user and 2) a PED frame to temporarily house a portable electronic device with a visual display unit that is positioned over the user's eyes to present visual stimuli, in which the visual stimuli is configured to evoke visual-event-related responses (VERPs) in the EEG signals exhibited by the user acquired by the sensor unit.

The head-mountable EEG device is configured to be worn on a user's head that allow users to couple and decouple a portable electronic device such as a handheld portable electronic device (e.g., temporarily integrates the separate devices into a single unit). Portable electronic device can be, for example, a portable media player, cellular telephone such as smartphones, internet-capable device such as minipads or tablet computers, personal organizer or digital assistants (“PDAs”), any other portable electronic device, or any combination thereof. In one embodiment of the present invention, the portable electronic device can be a device that has the combined functionalities of a portable media player and a cellular telephone.

One aspect of the invention relates to physically coupling (e.g., mechanically) the portable electronic device to the head-mounted EEG device such that the portable electronic device can be worn on the user's head. In some embodiments, the head-mounted EEG device may include a PED frame that supports, secures, and carries the portable electronic device (e.g., physically integrated as a single unit). The PED frame may also help place a display of the portable electronic device relative to a user's eyes when the integrated system is worn on the user's head. In one example, the PED frame helps define a docking area for receiving and retaining the portable electronic device.

Another aspect of the invention relates to operatively coupling (e.g., electronically) the portable electronic device to the head-mounted EEG device such that the portable electronic device and head-mounted EEG device can communicate and operate with one another. The head-mounted EEG device may include, for example, interface mechanisms that enable communication and operability between the portable electronic device and the head-mounted EEG device. The interface mechanisms may, for example, include electrical mechanisms such as connectors or chips that provide wired or wireless communications. In some embodiments, the head-mounted EEG device may include a connector that receives a corresponding connector of the portable electronic device. The connector may, for example, be located within a docking area of the head-mounted EEG device such that the portable electronic device operatively connects when the portable electronic device is placed within the docking area.

The interface mechanisms may also include optical interface mechanisms, such as lenses, etc., that provide optical communications for proper viewing of a display of the portable electronic device. For example, the optical interface mechanism can be an adjustable focus lens to enlarge or magnify images displayed on the portable electronic device.

Another aspect of the invention relates to allowing each device to extend its features and/or services to the other device for the purpose of enhancing, increasing and/or eliminating redundant functions between the head-mounted EEG device and the portable electronic device physically and/or operatively coupled thereto. In some embodiments, the head-mounted EEG device utilizes components of the portable electronic device while in other embodiments; the portable electronic device utilizes components of the head-mounted EEG device. For example, the head-mounted EEG device does not include a main viewing display screen and instead utilizes the screen of the portable electronic device to act as the main or primary display when the portable electronic device is coupled thereto. Further, the portable electronic device may have a processor that processes the EEG signal acquired from the user.

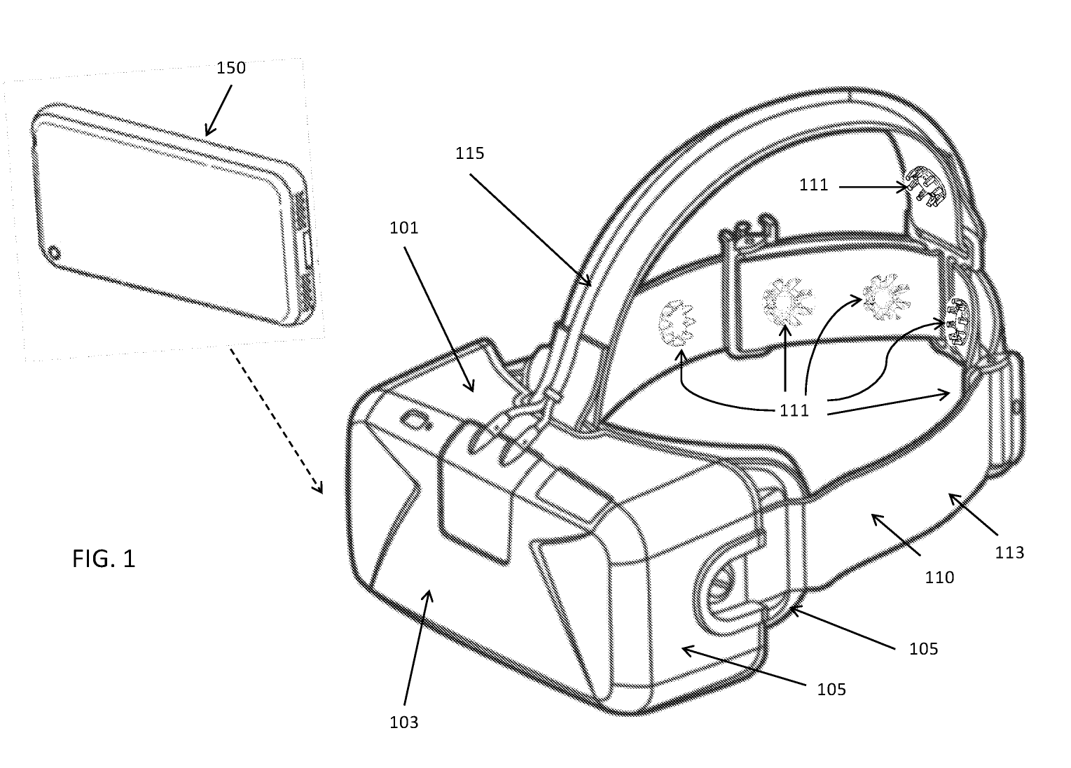

Embodiments of the invention are discussed below with reference to FIGS. 1-7. However, those skilled in the art will readily appreciate that the detailed description given herein with respect to these figures is for explanatory purposes, as the invention extends beyond these limited embodiments.

FIG. 1 shows a simplified diagram of a head-mounted EEG display system 100, in accordance with one embodiment of the present invention. The head-mounted EEG system 100 can include PED frame 101 and a sensor unit 110 to acquire electroencephalogram (EEG) signals from one or more EEG sensors 111 arranged to acquire EEG signals from the head of a user. A portable electronic device 150 that is a separate device can be temporarily coupled together to form an integrated unit, which can be worn on a user's head to monitor the electrical brain activity associated with visual field stimulation. The PED frame 101 may be supported on a user's head in a variety of ways including for example, ear support bars as in glasses, headbands as in goggles, helmets, straps, hats and the like. The sensor unit can be integrated into the support bars or headbands. These interfaces can monitor and record non-invasive, high spatiotemporal resolution brain activity of unconstrained, actively engaged human subjects.

FIG. 1 shows one embodiment with a head-mounted EEG system having a sensor unit comprising a headband 113 that includes a plurality of electrode sensors 111 to provide contact or near contact with the scalp of a user. In lieu of a headband, sensor units can reside on other structure such as ear support bars. Sensors 111 can circumnavigate headband to record EEG signals across, for example, the parieto-occipital region of the brain. In the case of an ear support bar, it can measure around the temple and ear of the user. Multiple headbands 113 can be used to secure the head-mounted display EEG device 101 near the front of the user's head and the sensors 111 to measure different cross sections of the head. Sensors can be permanently attached to headband or can be removable/replaceable, for example, plug-in sockets or male/female sockets. Each sensor can be of sufficient length to reach the scalp, spring-loaded or pliable/flexible to “give” upon contact with the scalp, or contactless to capture EEG signals without physical contact. Sensors 111 may have rounded outer surfaces to avoid trauma to the wearer's head, more preferably flanged tips to ensure safe consistent contact with scalp. Sensors 111 may be arranged in one or more linear rows provided in spaced relation along headband. The headband 113 may be made of fabric, polymeric, or other flexible materials that may provide additional structure, stiffness, or flexibility to position the display on the portable electronic device proximal to the eyes of the user and the sensor unit 110 to contact the scalp of the user.

Any of a variety of electrodes known for use with EEG can be used with the present device. In one embodiment, the sensor unit 110 can comprise one electrode or multiple electrodes 111. Electrode sensors 111 can be of varying sizes (e.g., widths and lengths), shapes (e.g., silo, linear waves or ridges, pyramidal), material, density, form-factors, and the like to acquire strongest signal and/or reduce noise, especially to minimize interference of the hair. The sensors may be interconnected to capture a large area or independently in multiple channels to capture an array of EEG signals from different locations. FIG. 1 illustrates discrete placement of independent electrodes sensors 111 comprising conductive spiked sensors across the occipital region and parietal region of the head where they may encounter hair. In one embodiment, electrodes are made of foam or similar flexible material having conductive tips or conductive fiber to create robust individual connections without potential to irritate the skin of the user (e.g., “poking”).

Electrode sensors 111 utilized in the invention can either be entirely conductive, mixed or associated with or within non-conductive or semi-conductive material, or partially conductive such as on the tips of electrodes. For example, in certain embodiments, the conductive electrodes are woven with or without non-conductive material into a fabric, net, or mesh-like material, for example, the headband, to increase flexibility and comfort of the electrode or embedded or sewn into the fabric or other substrate of the head strap, or by other means. The EEG sensors 111 can be wet or dry electrodes. Electrode sensor material may be a metal such as stainless steel or copper, such as inert metals, like, gold, silver (silver/silver chloride), carbon, tin, palladium, and platinum or other conductive material to acquire an electrical signal, including conductive gels and other such composition. The electrode sensors 111 can also be removable, including for example, a disposable conductive polymer or foam electrode. The electrode sensors 111 can be flexible, preshaped or rigid, and in any shape, for example, a sheet, rectangular, circular, or such other shape conducive to make contact with the wearer's skin. For example, electrode can have an outfacing conductive layer to make contact with the scalp and an inner connection to connect to the electronic components of the invention. The invention further contemplates electrode sensors 111 for different location placements. For example, electrodes for the top of the head may encounter hair. Accordingly, electrodes on the ends of “teeth”, clips or springs may be utilized to reach the scalp of the head through the air. Examples of such embodiments as well as other similar electrodes on headbands are discussed in U.S. patent application Ser. No. 13/899,515, entitled EEG Hair Band, incorporated herein by reference.

The present invention contemplates different combinations and numbers of electrodes and electrode assemblies to be utilized. As to electrodes, the amount and arrangement thereof both can be varied corresponding to different demands, including allowable space, cost, utility and application. Thus, there is no limitation. The electrode assembly typically will have more than one electrode, for example, several or more electrode each corresponding to a separate electrode lead, although different numbers of electrodes are easily supported, in the range of 2-300 or more electrodes, for example.

The size of the electrodes on the headband may be a trade between being able to fit several electrodes within a confined space, and the capacitance of the electrode being proportional to the area, although the conductance of the sensor and the wiring may also contribute to the overall sensitivity of the electrodes.

It is expected that one or more electrodes will be used as a ground or reference terminal (that may be attached to a part of the body, such as an ear, earlobe, neck, face, scalp, or alternatively or chest, for example) for connection to the ground plane of the device. The ground and/or reference electrode can be dedicated to one electrode, multiple electrodes or alternate between different electrodes.

The present technology utilizes electroencephalogram (EEG)-based brain sensing methods, systems, and devices for visual-field examination by using EEG to associate the dynamics of visual-event-related responses (VERPs) with visual field defects. In one embodiment, the invention uses solid state visual-event-related responses (SSVERP), in which the use of rapid flickering stimulation can produce a brain response characterized by a “quasi-sinusoidal” waveform whose frequency components are constant in amplitude and phase, the so-called steady-state response. Steady-state VERPs have desirable properties for use in the assessment of the integrity of the visual system.

Portable electronic device 150 may be widely varied. For example, portable electronic device 150 may be configured to provide specific features and/or applications for use by a user. Portable electronic device 150 may be a lightweight and small form factor device so that it can easily be supported on a user's head. In most embodiments, the portable electronic device includes a display for viewing image-based content.

In one embodiment of the present invention, portable electronic device 150 may be a handheld electronic device such as a portable media player, cellular telephone, internet-capable device, a personal digital assistant (“PDA”), any other portable electronic device, or any combination thereof. In another embodiment of the present invention, portable electronic device 150 can be a device that has the combined functionalities of a portable media player and a cellular telephone.

The PED frame 101 may be configured to receive and carry portable electronic device 150. In some embodiments, PED frame 101 may include a support structure 105 that supports and holds the portable electronic device 150 thereby allowing portable electronic device 150 to be worn on a user's head (e.g., glasses/goggles form factor). The support structure 105 may for example be configured to be situated in front of a user's face. As a result, screen of the portable electronic device 150 may be oriented towards the user's eyes when head-mounted EEG display system 100 (the PED frame 101 including the portable electronic device 150) is worn on the user's head.

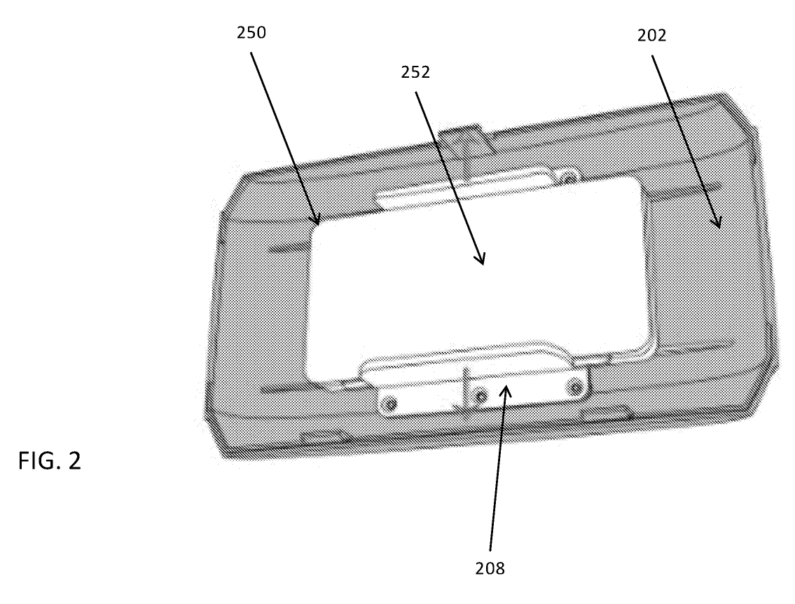

The support structure 105 may define or include a docking member 202 (as shown in FIG. 2) for receiving and retaining, securing or mounting the portable electronic device 250. The docking member 202 may be widely varied. The docking member 202 defines an area into which a portion or the entire portable electronic device 250 may be placed. The docking member 202 may also include one or more retention features 204 for holding and securing the portable electronic device 250 within the docking area 202. The docking member 202 may be defined by walls that surround some portion of the portable electronic device 250 (e.g., exterior surfaces). The retention features 204 may for example include rails, tabs, slots, lips, clips, channels, snaps, detents, latches, catches, magnets, friction couplings, doors, locks, flexures, and the like.

In some embodiments, support structure can include an adjustable mating mechanism such that the portable electronic device can fit regardless of the size of the device or the presence or absence of a case used for the device (e.g., soft or hard case). For example, the shape and dimensions of the cavity may be physically adjusted so as to fit different portable electronic devices. Moreover, the cavity may be oversized and include a separate insert for placement therein. In some cases, the cavity may provide the retaining structure by being dimensioned to snuggly receive the portable electronic device (e.g., friction coupling). In some cases, the cavity may include a biasing element such as flexures or foam that conforms and cradles the portable electronic device when contained within the cavity. The material can also be suitable for pooling heat away from the portable electronic device. In some cases, the slot may include a door that locks the portable electronic device within the cavity. The retaining feature may also act as a bezel that covers or overlays select portions of the portable electronic device 204 to form or define the viewing region. The docking member 202 is configured to orient the display screen 253 (towards the eyes of the user) in the correct position for viewing relative to a user's eyes (e.g., in front of the users eyes as well as some of the distance from the user's eyes).

The head-mounted EEG display system 100 can include a communication interface 115 that provides data and/or power communications between the portable electronic device 150 and the head-mounted EEG display system. The communication interface may be wired or wireless. In some embodiments, the head-mounted EEG device 100 may include a connector that mates with a corresponding connector of the portable electronic device when the portable electronic device is placed within the docking area 103.

Generally, the communication session begins when the portable electronic device 150 is coupled together and powered up. For example, based on default settings, the portable electronic device 150 may be configured for close up head-mounted viewing (either directly or via instructions from the head-mounted EEG device 100). Further, input devices, output devices, sensors, and other electrical systems on both devices may be activated or deactivated based on the default settings. Alternatively, the user may be prompted with a control menu for setting up the system when they are operatively coupled together via the communication interface 115. In line, the communication session terminates upon disconnection with the portable electronic device. The device can be also manually deactivated by the user or automatically deactivated, for example, if no user selection is received after a certain period of time.

The system may include a detection mechanism for alerting the portable electronic device 204 that it has been mounted or is otherwise carried by PED frame. If user preferences are used, the user may be able to make adjustments as needed. Since adjustments may be difficult for the user, in some cases, the system and/or portable electronic device may include mechanisms for automatically configuring the image location and size. For example, either device may include sensors for detecting the distance to the eyes and the position of the eyes. As should be appreciated, each user's eyes are oriented differently. For example some eyes are located close together while others are more spread out. The optimal viewing positions of the displayed images can be determined and then the viewing positions can be adjusted. The same can be done for resolution. Although, allowing the user to adjust resolution may be beneficial as this is a more difficult measurement to make since eyes can focus differently. By way of example, the portable electronic device and/or the PED frame may include cameras that can reference where the eyes are located relative to the PED frame.

The resolution of the displayed image frames can also be adjusted in a similar manner. However, because each user's eyes focus differently, it may be beneficial to allow the user to manually adjust the resolution, as this is a more difficult measurement to make. The size and possibly the resolution of the image-based content being displayed on the screen may be adjusted for close up viewing (e.g., via the detection mechanism or the connection interface). When coupled, the distance of the display screen relative to the user's eyes may be widely varied. In small form factor head mountable devices (e.g., low profile), the display screen of the portable electronic device 150 may be placed fairly close to the user's eyes. The placement of the display screen may be controlled by the surfaces of mounting region 208 and more particularly the walls of the cavity 212. In addition, the image-based content may be displayed (e.g., by electrical adjustment of the portable electronic device or the image, respectively) in a viewing region that is configured the full size or configured smaller than the actual screen size (e.g., due to how close it is placed to the user's eyes) and/or the resolution may be increased/decreased relative to normal portable electronic device viewing to provide the best close up viewing experience. In one implementation, the viewing region is configured to fill the entire field of view of the user to test the boundaries of the user's field of vision. In another implementation, the viewing region is configured to be less than the field of view of the user.

In one embodiment, the head-mounted EEG display system may include a sensing mechanism for alerting the portable electronic device 400 that the device has been coupled to the head-mounted display EEG device 300. As a result, portable electronic device 400 can be activated. By way of example, the sensing mechanism may be an electrical connection, a sensor such as a proximity sensor or IR detector, and/or the like. The sensing mechanism may be used instead of or in combination with the communication interface to assist the devices into adjusting to the user.

In one embodiment, the displayed content may be split into multiple images frames, e.g., binocular display. For example, the displayed content may be split into two image frames (e.g., a left and right image frame for the left and right eye of the user). With two image frames, the system can test separately the right eye and the left eye, or perform stereoscopic imaging. Stereoscopic imaging attempts to create depth to the images by simulating the angular difference between the images viewed by each eye when looking at an object, due to the different positions of the eyes. This angular difference is one of the key parameters the human brain uses in processing images to create depth perception or distance in human vision. In one example, a single source image is processed to generate left image data and right image data for viewing. The timing or image characteristics of the dual image frames relative to one another may be varied to provide an enhanced viewing effect. This can be accomplished by the portable electronic device and/or the head-mounted EEG system depending on the needs of the system.

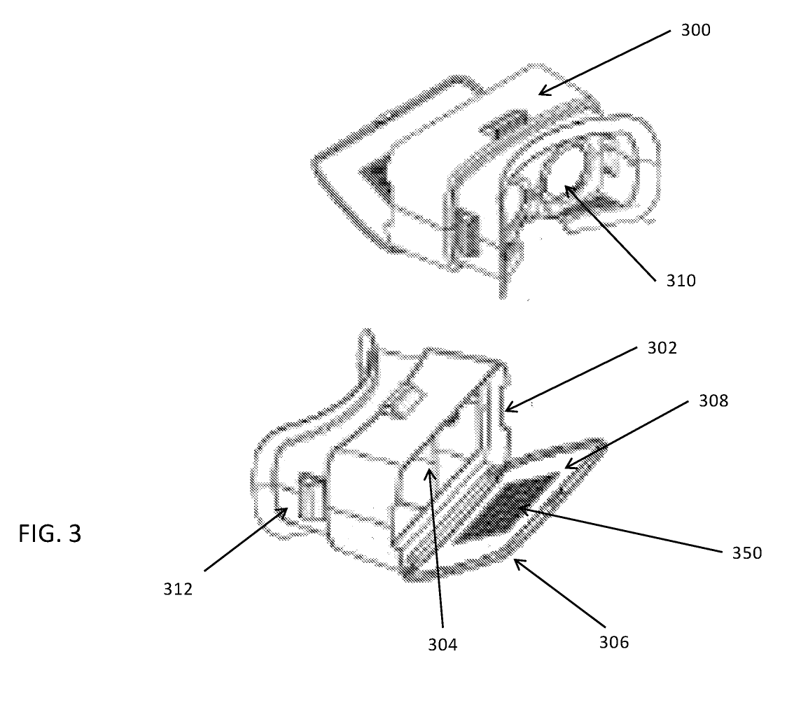

FIG. 3 shows opposing views of an open PED frame 300 in accordance with one embodiment of the present invention. PED frame 300 shown in FIG. 3 may generally correspond to the head-mounted EEG display system described in FIG. 1 without the sensor unit. PED frame 300 receives a portable electronic device 350 having a display screen. That is, portable electronic device 350 may be coupled to PED frame (as shown in FIG. 3) and positioned for user to view the display.

The PED frame 300 may be widely varied. In one embodiment, the PED frame 300 includes a support structure and a docking member 306. The support structure and the docking member may be one unit or separate, with the docking member acting as a lid to the support structure. The PED frame 300 may for example have four walls 302 of the support structure contoured to the outer edge of the portable electronic device 350 and a docking member as the fifth wall supporting the portable electronic device. In some cases, the PED frame 302 may only include walls that surround multiple but not all sides of the portable electronic device 350 (e.g., at least two sides, three sides, fours sides, five sides,). Additional walls 304 may be used, for example to separate the viewing of the left and right eye. In any of these implementations, the walls may include open areas depending on the needs of the system. Alternatively, the PED frame 300 may be formed with corners that match the corners of the portable electronic device 350.

PED frame can be constructed into any suitable shape. In one example, the user facing side takes the shape of the eyes and nose area of the face and the other sides are substantially planar surfaces. As another example, the left and right side of the PED frame can be curved surfaces that generally follow the contours of a user's face.

PED frame 300 can be formed from any suitable material or materials. In some embodiments, the PED frame 300 can be formed from lightweight materials that afford user comfort (e.g., plastic) while maintaining strength to support a portable electronic device. In some embodiments, the PED frame 300 can be formed from a material capable of withstanding impacts or shocks to protect the components of the head-mounted EEG display system. Examples of materials include composite material, glass, plastic (ABS, polycarbonate), ceramic, metal (e.g., polished aluminum), metal alloys (e.g., steel, stainless steel, titanium, or magnesium-based alloys), or any other suitable material. In some embodiments, the outer surface of PED frame 300 can be treated to provide an aesthetically pleasing finish (e.g., a reflective finish, or added logos or designs) to enhance the appearance of system.

PED frame 300 may be a skeletal structure with minimal structure such as walls thereby keeping it light-weight and/or it may be configured more like a housing that can enclose various components. PED frame 300 may include support structure 302, which helps form the side surface of the PED frame 300. PED frame 300 may also include a front panel and/or a back panel that can be integral with or coupled to support structure 302 to form the front and back surfaces of PED frame 300. The back panel can also act as docking member. Thus, support structure 302, front panel, back panel 306 can cooperate to form the outer structure of head-mounted display EEG device 300.

Support structure 302, front panel and back panel 306 can be formed from any suitable material as mentioned above. In some embodiments, the three structures are formed from similar materials. In other embodiments, the three structures are formed from dissimilar materials. Each has needs that may be taken into account when designing the head-mounted display EEG device. For example, the support structure may be formed from a structure material with a structural configuration thereby providing central support to the PED frame 300 while the front and back panels may be formed a material capable of withstanding impacts or shocks to protect the components of head-mounted EEG display system.

The PED frame 300 can include any suitable feature for improving the user's comfort or ease of use when the portable electronic device is coupled to the head-mounted display EEG device. FIGS. 1 and 3 shows illustrative features for exemplary head-mounted display EEG devices. FIGS. 1 and 3 shows a face mask or skirt 105/312 on at least a lower portion of the device. Mask/skirt 312 can be made from any material relatively comfortable such as rubber, plastic, foam or material that can deform or substantially comply with the user's face (e.g., nose) thus improving the user's comfort, or combinations thereof. For example, in some cases, foam is placed at the location where the frame engages the nose (e.g., nose cut out). In other cases, the foam is placed continuously or selectively across the entire bottom edge that engages the nose and face. Still further, the foam may be placed continuously or selectively across the entire edge of the frame that engages the nose and face (upper, side and lower portions). The structural portion of mask/skirt adjoining foam and support structure can be made of plastic or rubber to add rigidity to mask/skirt. In fact, in some implementations, because the material is deformable, the bottom surface of the head-mounted display EEG device can be flat when the device is not being worn (e.g., no nose cut out).

Mask/skirt 312 can be used to prevent ambient light from entering between the user's face and the head-mounted display EEG device (e.g., provides a seal between the frame and the user's face). Additionally, mask/skirt 312 can be used to reduce the load on the user's nose because the portable electronic device can be relatively heavy. In some cases, mask/skirt 312 can serve to increase a user's comfort with the PED frame by helping to center the frame on the user's face. Alternatively or additionally, the PED frame may include a shroud (not shown) that helps enclose the viewing experience. The shroud may, for example, be one or more shaped panels that fill and/or cover the air gaps normally found between the frame and the user's face. In fact, the deformable material may be applied to the shroud.

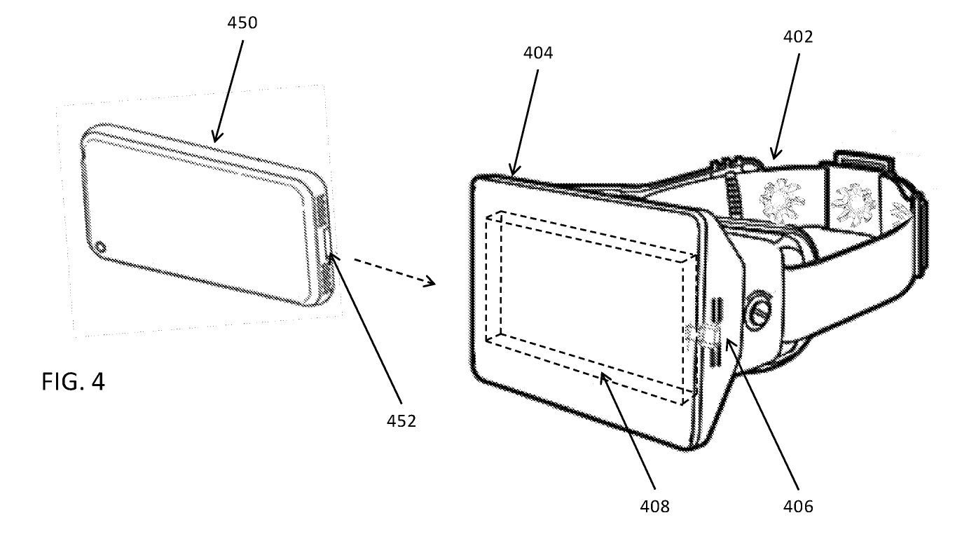

The manner in which the portable electronic device is placed within the docking member may be widely varied. In one implementation, the portable electronic device may be rotated or dropped into the docking member (e.g., by inserting a first end into the docking member and thereafter rotating the docking member closed as shown in FIG. 3). In another implementation, the portable electronic device may be press fit into the docking member (e.g., by pushing the portable electronic device into the shaped cavity as shown in FIG. 2). In yet another implementation, the portable electronic device may be slid into the cavity (e.g., through a slot in one of its sides as shown in FIG. 4).

Head-mounted EEG display system can include a variety of features, which can be provided by one or more electronic subassemblies, when they are connected and in communications with one another. For example, each device may include one or more of the following components: processors, display screen, controls (e.g., buttons, switches, touch pads, and/or screens), signal amplifiers, A/D (and/or D/A) converters, camera, receiver, antenna, microphone, speaker, batteries, optical subassembly, sensors, memory, communication circuitry or systems, input/output (“I/O”) systems, connectivity systems, cooling systems, connectors, and/or the like. If activated, these components may be configured to work together or separately depending on the needs of the system. In some cases, features may be turned off entirely if not needed by the system.

Electronic subassemblies can be configured to implement any suitable functionality provided by head-mounted display EEG device 300. The one or more subassemblies may be placed at various locations within or outside of the head-mounted display EEG device. For example, the electronic subassemblies may be disposed at internal spaces defined by PED frame or within the sensor unit (without interfering with the internal space provided for the portable electronic device or the EEG acquisition). In one example, they are placed at the lower sections on the right and left of the nose support region of the PED frame. Additionally or alternatively, the PED frame may form enclosed portions that extend outwardly thereby forming internal spaces for placing the electronic subassemblies. In yet another example, the headband encases electronic subassemblies.

In one embodiment, the system is configured to utilize the processing capability of the portable electronic device to coordinate the visual stimulus and the acquisition of the brain activity of the user. In yet a further, or alternative embodiment, the EEG display device will have a separate data-processing unit.

The data processing unit can include a processor that can be in communication with portable electronic device. Processor can be connected to any component in the system, for example, via a bus, and can be configured to perform any suitable function, such as audio and video processing, and/or processing of EEG signals. For example, processor can convert (and encode/decode, if necessary) data, analog signals, and other signals (e.g., brain signals (e.g., EEG), physical contact inputs, physical movements, analog audio signals, etc.) into digital data, and vice-versa. Processor can also coordinate functions with portable electronic device, for example, initiate system activation, optimize settings of system, provide protocol for testing, label EEG signals to coordinate with the visual stimulus, transform EEG signals, artifact removal and signal separation, compare datasets recorded from user at different times and using different tests, and the like. In some embodiments, processor can receive user inputs from controls and execute operations in response to the inputs. For example, processor can be configured to receive sound from the microphone. In response to receiving the sound, processor can run the voice recognition module to identify voice commands. Processor can alternatively coordinate with portable electronic device to perform these functions. Alternatively, data processing can be implemented as one of various data processing systems, such as on a personal computer (PC), laptop, and system mobile communication device. In some implementations, the data processing unit can be included in the device structure that includes the wearable EEG sensor unit. To support various functions of the data processing unit, the processor can be included to interface with and control operations of the portable electronic device, the electronic subassemblies of the device and the memory unit.

Head-mounted display EEG device may include memory. Memory can be one or more storage mediums, including for example, a hard-drive, cache, flash memory, permanent memory such as read only memory (“ROM”), semi-permanent memory such as random access memory (“RAM”), any other suitable type of storage component, or any combination thereof. In some embodiments, memory can provide additional storage for EEG content and/or image-based content that can be played back (e.g., audio, video, test, and games). For example, when a user couples portable electronic device into head-mounted display EEG device, the user can select to play a diagnostic stored on EEG display device, or alternatively stored on the portable electronic device. In one embodiment, the portable electronic device will download an application or mobile app specific to the diagnostic. In response to the user selecting to run a diagnostic test, the test can be loaded or streamed to portable electronic device, which run the test on the user. In some embodiments, the test can be copied into memory on portable electronic device. The memory unit can store data and information, which can include subject stimulus and response data, and information about other units of the system, e.g., including the EEG sensor unit and the visual display unit, such as device system parameters and hardware constraints. The memory unit can store data and information that can be used to implement the portable EEG-based system, such as the acquired or processed EEG information.

Head-mounted display EEG device can include battery, which can charge and/or power portable electronic device when portable electronic device is coupled to head-mounted display EEG device. As a result, the battery life of portable electronic device can be extended.

Head-mounted display EEG device can include cooling system, which can include any suitable component for cooling down portable electronic device. Suitable components can include, for example, fans, pipes for transferring heat, vents, apertures, holes, any other component suitable for distributing and diffusing heat, or any combination thereof. Cooling system may also or instead be manufactured from materials selected for heat dissipation properties. For example, the housing of head-mounted display EEG device may be configured to distribute heat away from portable electronic device and/or the data-processing unit.

The system can include a communication interface that provides data and/or power communications between the portable electronic device and the head-mounted EEG display system. The communication interface may be wired or wireless.

As shown in FIG. 4, if wired, the head-mounted EEG display system may include a connector 406 that receives a corresponding connector 452 of the portable electronic device 450 when the portable electronic device 450 is supported/carried by the PED frame 404. In most cases, the connectors mate when the device is placed within the PED frame 404, and more particularly when placed within the cavity 408. By way of example, the connectors may mate as the portable electronic device is rotated, slid, or pressed into the PED frame 404. The connectors may be male/female. For example, the portable electronic device 450 may include a female connector while the PED frame 404 may include a male connector. In this particular case, the male connector is inserted into the female connector when the devices are coupled together. The connectors may be widely varied. The connectors may be low profile connectors. The connectors may for connectors generally used by portable electronic devices such as USB (including mini and micro), lightning, FireWire, and/or proprietary connections, such as a 30-pin connector (Apple Inc.). In some cases, the cavity/connector combination may generally define a docking station for the portable electronic device.

Alternatively or additionally, the data and/or power connection can be provided by a wireless connection. Wireless connections may be widely varied. For example, the devices may each include a wireless chip set that transmits and/or receives (transceiver) the desired signals between the devices. Examples of wireless signal protocols include Bluetooth™ (which is a trademark owned by Bluetooth Sig, Inc.), 802.11, RF, and the like. Wireless connections may require that wireless capabilities be activated for both the head-mounted display EEG device and the portable electronic device. However, such a configuration may not be possible or may be intermittent when the devices are being used in certain locations as, for example, on an airplane.

In some embodiments, head-mounted display EEG device can include I/O units such as connectors or jacks, which can be one or more external connectors that can be used to connect to other external devices or systems (data and/or power). Any suitable device can be coupled to portable electronic device, such as, for example, an accessory device, host device, external power source, or any combination thereof. A host device can be, for example, a desktop or laptop computer or data server from which portable electronic device can provide or receive content files. Persons skilled in the art will appreciate that connector can be any suitable connector. For example, the head-mounted display EEG device can also include one or more I/O units that can be connected to an external interface, source of data storage, or for communicating with one or more servers or other devices using any suitable communications protocol. Various types of wired or wireless interfaces compatible with typical data communication standards can be used in communications of the data processing unit with the EEG sensor unit and the portable electronic device and/or other units of the system, e.g., including, but not limited to, Universal Serial Bus (USB), IEEE 1394 (FireWire), Bluetooth™ (which is a trademark owned by Bluetooth Sig, Inc.), Wi-Fi (e.g., a 802.11 protocol), Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/LTE cellular communication methods, Ethernet, high frequency systems (e.g., 900 MHz, 2.4 GHz, and 5.6 GHz communication systems), infrared, TCP/IP (e.g., any of the protocols used in each of the TCP/IP layers), HTTP, BitTorrent, FTP, RTP, RTSP, SSH, and parallel interfaces, any other communications protocol, or any combination thereof, can be used to implement the I/O unit. The I/O unit can interface with an external interface, source of data storage, or portable electronic device to retrieve and transfer data and information that can be processed by the processor, stored in the memory unit, or exhibited on the output unit.

Communications circuitry can also use any appropriate communications protocol to communicate with a remote server (or computer). The remote server can be a database that stores various tests and stimuli (and applications for running same) and/or any results. When head-mounted display EEG device is connected to the remote server, content (e.g., tests, images, games, content, videos, previous results of history, processed EEG, training protocol, instructions, etc.) can be downloaded to or uploaded from portable electronic device or head-mounted display EEG device for use. The content can be stored on portable electronic device, head-mounted display EEG device, or any combination thereof. In addition, the stored content can be removed once use has ended.

In some embodiments, the PED frame and the sensor unit may provide additional features for the head-mounted EEG display system. In one example, the head-mounted EEG system can provide additional functionality to the portable electronic device.

In addition, the head-mounted EEG system can include a battery to extend the life of the portable electronic device. Furthermore, the head-mounted EEG display system can include a cooling system for cooling down the portable electronic device. Persons skilled in the art will appreciate that any other suitable functionality may be extended including additional circuitry, processors, input/output, optics, and/or the like.

In some embodiments, head-mounted EEG display system can provide controls that can allow the user to control the portable electronic device while wearing system. Controls can control any suitable feature and/or operation of system and/or the portable electronic device. For example, controls can include navigation controls, display controls, volume controls, playback controls, or any other suitable controls. Controls can be located on the side surfaces, front surface, top surface, headband or ear support bars, or any other accessible location on the periphery of head-mounted display EEG device 300.

Any suitable type of controls can be used, such as, for example, wheels, dials, buttons, switches, sliders, and touch sensors. In some embodiments, a touch sensor can be used to measure the response of the user. As an example, a longitudinal touch sensor can be placed along headband or support bar. As still another example, touch sensors can also be used for display controls (e.g., brightness and contrast, enlarge/shrink, camera zoom, or any other suitable display control). These controls may match or mimic the controls found on the portable electronic device.

In some implementations, for example, the disclosed techniques include using SSVERP and brain-computer interfaces (BCIs) to bridge the human brain with computers or external devices. By detecting the SSVERP frequencies from the non-invasively recorded EEG, the users of SSVERP-based brain-computer interface can interact with or control external devices and/or environments through gazing at distinct frequency-coded targets. For example, the SSVERP-based BCI can provide a promising communication carrier for patients with disabilities due to its high signal-to-noise ratio over the visual cortex, which can be measured by EEG at the parieto-occipital region noninvasively.

Remote control can be connected to head-mounted display EEG device or the portable electronic device using any suitable approach. For example, remote control can be a wired device that is plugged into a connector. As another example, remote control can be a wireless device that can transmit commands to the portable electronic device and head-mounted display EEG device via a wireless communications protocol (e.g., Wi-Fi, infrared, Bluetooth™ or any combination thereof). As still yet another example, remote control can be a device that is capable of both wired and wireless communications. The user may use remote control to navigate the portable electronic device and to control the display, volume, and playback options on the portable electronic device.

As further illustrated in FIG. 3, the PED frame 300 may include an optical subassembly 310 for helping properly display the one or more image frames to the user. That is, the optical subassembly 310 may help transform the image frame(s) into an image(s) that can be viewed by the human eye. Optical subassembly may for example focus the images from the respective image frame(s) onto the user's eyes at a comfortable viewing distance.

The optical subassembly 310 may be disposed between the display screen and the user's eyes. The optical subassembly 310 may be positioned in front of, behind or within the opening that provides viewing access to the display screen. The PED frame 300 may support the optical subassembly 310. For example, it may be attached to the PED frame 300 via any suitable means including for example screws, adhesives, clips, snaps, and the like.

The optical subassembly 310 may be widely varied. The optical subassembly 310 may include various optical components that may be static or dynamic components depending on the needs of the system. The optical components may include, for example, but not limited to lenses, light guides, light sources, mirrors, diffusers, and the like. The optical subassembly 310 may be a singular mechanism or it may include dual features, one for each eye/image area. In one implementation, the optical subassembly 310 can be formed as a panel that overlays the access opening. The panel may be curvilinear and/or rectilinear. For example, it may be a thin flat panel that can be easily carried by the PED frame 300 and easily supported on a user's head. If dynamic, the optical subassembly 310 may be manually or automatically controlled.

Electrooculogram (EOG) methods of the disclosed technology can be utilized to successfully identify fixation losses and allow identification of unreliable mfSSVERP signals to be removed from further analyses. For example, from the earlier implementations of an mfSSVERP technique for assessment of visual field loss, the strength of mfSSVERP in one of the five participates failed to accurately reflect the mimicked visual deficit. The reason, for example, may be attributed to the absence of proper gaze fixation during the examination based on the patient's self-report. In order to assure matching of SSVERP signals to corresponding visual field locations, subjects need to remain fixating on the central target location during the testing. Due to the short duration of testing trials, this can be achieved in most subjects, yet, the disclosed technology includes a mechanism to identify and exclude unreliable EEG signals produced by fixation losses. This is especially relevant in portable testing that may be performed without supervision.

In some embodiments, for example, the disclosed portable VERP systems can include an electrooculogram (EOG), electromyogram (EMG), electrocardiography (ECG), and/or electro¬dermal activity (EDA) unit. In one example embodiment, the invention further comprises an EOG unit that can include two or more dry and soft electrodes to be placed proximate the outer canthus of a subject's eyes (e.g., one or more electrodes per eye) to measure corneo-retinal standing potentials, and are in communication with a signal processing and wireless communication unit of the EOG unit to process the acquired signals from the electrodes and relay the processed signals as data to the data processing unit of the portable system. In some implementations, the electrodes of the EOG unit can be in communication with the EEG unit or visual display unit to transfer the acquired signals from the outer canthus-placed electrodes of the EOG unit to the data processing unit.

For example, in order to remove unreliable EEG signals occurring from fixation losses, the disclosed techniques can concurrently monitor subjects' electrooculogram (EOG) signals to evaluate the gaze fixation. By placing the dry and soft electrodes of the EOG unit to the outer canthus of the eyes, the electric field changes associated with eye movements, e.g., such as blinks and saccades, can be monitored. There is a linear relationship between horizontal and vertical EOG signals and the angle of eye rotation within a limited range (e.g., approximately 30°). This relationship can be used in determining the exact coordinates of eye fixations on a visual display. In some implementations, a calibration sequence can be used at the start of recording to determine the transformation equations. Accordingly, for example, an EOG-guided VERP analysis can be implemented to automatically exclude the EEG segments where the subjects do not gaze at the center of the stimulation. To record EOG signals, four prefrontal electrodes can be switched to record the EOG signals. In one example in which the EOG unit includes four electrodes, two electrodes can be placed below and above the right eye and another two will be placed at the left and right outer canthus. The EOG unit can be used to assess the accuracy of the portable VERP system by identifying potentially unreliable EEG signals induced by loss of fixation. For example, the data processing unit can process the acquired signals from the EOG unit electrodes with the EEG data acquired from the EEG unit to identify unreliable signals, which can then be removed from the analysis of visual field integrity. For example, the data processing unit can execute analytical techniques to provide signal source separation. Additionally, or alternatively, for example, the disclosed portable VERP systems can include an eye-tracking unit to monitor losses of fixation, e.g., and can further provide a reference standard. For example, the eye-tracking unit can be included, integrated, and/or incorporated into the visual display unit (e.g., exemplary head-mounted EEG display), for example.

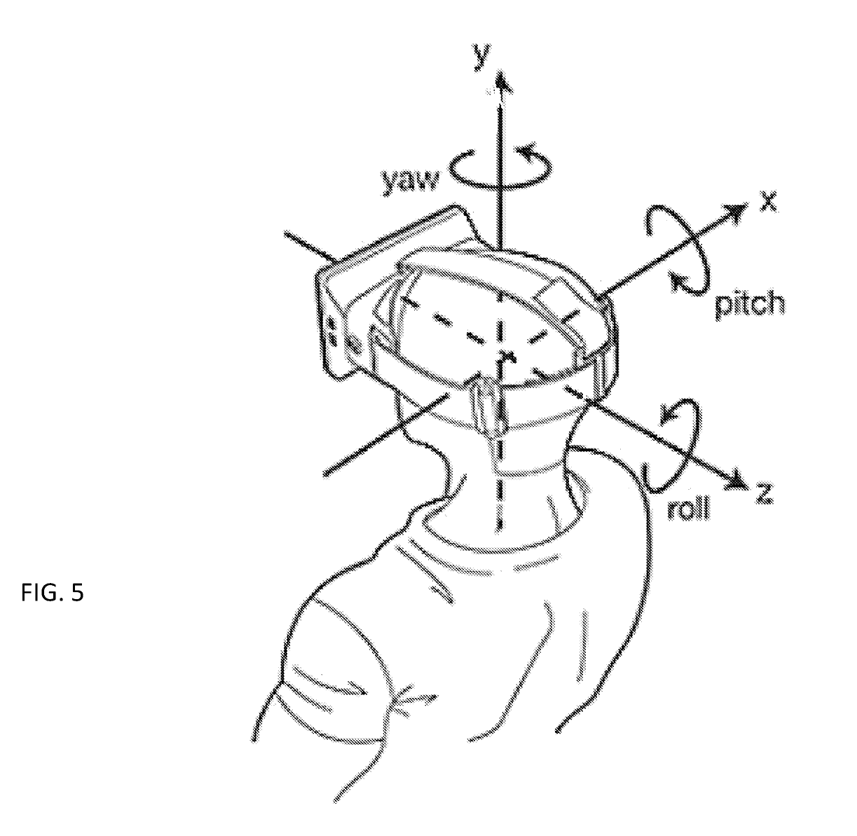

In some embodiments, the system can include one or more sensors incorporated on the head-mounted EEG display system 100 and/or use sensors available on the portable electronic device 150 to detect various signals. Suitable sensors can include, for example, ambient sound detectors, proximity sensors, accelerometers, light detectors, cameras, and temperature sensors. An ambient sound detector can aid the user with hearing a particular sound. For example, accelerometers and gyroscopes on the head-mounted EEG display system 100 (see FIG. 5) and/or the portable electronic device can be used to detect the user's head movements. In this example, the head-mounted EEG display system 100 can associate a particular head movement with a command for controlling an operation of the system 100. As yet another example, the head-mounted EEG display system 100 can utilize a proximity sensor on one or both of the system and portable electronic device to detect and identify the relationship between the two devices or to detect and identify things in the outside environment. As yet another example, the head-mounted EEG display system 100 can utilize a microphone on one or both of the head-mounted display EEG device and portable electronic device to detect and identify voice commands that can be used to control the portable electronic device 150. As yet another example, the head-mounted EEG display system 100 can utilize a camera on one or both of the head-mounted display EEG device and portable electronic device to capture images and/or video. The image-based content may for example be viewed on the display of the head-mounted EEG display system. In one embodiment, the image-based content may be viewed in addition or alternatively to image-based media content playing on the display. In one example, the captured content may be viewed in a picture in picture window along with the media based content.

Head-mounted display EEG device may also include a camera region. The camera region may represent a camera that is integrated with the head-mounted display EEG device. An integrated camera may be used in place of or in conjunction with a camera on the portable electronic device. In cases where the portable electronic device includes a camera, and there is a desire to reduce redundancies (e.g., thereby reducing weight, complexity and cost), PED frame can have openings aligned with one or more cameras of the portable electronic device when the portable electronic device is situated inside device. The camera hole can allow the camera on the portable electronic device to capture image-based content of the user's surroundings. For example, camera(s) can be used when head-mounted display EEG device 300 is worn on the user's head to provide image-based content to the user. Alternatively, if portable electronic device has a camera-facing user, camera can be used to measure one or both eyes of the user, e.g., for measuring features of the eye such as placement, proximity to each other, identity of user such as a retinal scan or facial feature scan.

Head-mounted display EEG device may include speakers. Speakers can be located at various locations on head-mounted display EEG device to enhance the user's viewing experience. For example, speakers can be placed around some or all of the periphery (e.g., sides, top, and/or bottom) of frame. As another example, speakers can be integrated into headband or strap, which can be located at the user's ear level. As still another example, speakers can be placed on eyeglass temples, which can fit over or behind the user's ears. Speakers can include a variety of different types of speakers (e.g., mini speakers, piezo electric speakers, and the like), and/or haptic devices. Speakers can also be utilized to measure auditory evoked potentials, and deterioration of auditory nerves.

Haptic devices (e.g., buzzers, or vibrators) can work alone or in combination with speakers. In some cases, the speakers may serve as haptic components. Similarly to the speakers, haptics can be placed around some or all of the periphery (e.g., sides, top, and/or bottom) of frame. As another example, haptics can be integrated into strap 310, which can be located at the user's ear level. As still another example, speakers can be placed on eyeglass temples, which can fit over or behind the user's ears. Haptic devices can interface with the user through the sense of touch by applying mechanical stimulations (e.g., forces, vibrations, and motions). For example, while a user is watching image-based content, haptic devices can be configured to provide an enhanced surround sound experience by providing impulses corresponding to events in the image-based content. As an illustrative example, the user may be watching a movie that shows an airplane flying on the left of the screen. Haptic devices can produce vibrations that simulate the effect (e.g., sound effect, shock wave, or any combination thereof) of the airplane. For example, a series vibration may be provided along the left temple from front to back to simulate the airplane flying to the left and rear of the user. Speakers can also be used in this manner.

After coupling the portable electronic device to the head-mounted display EEG device, the protocol under which devices communicate may be widely varied. Any suitable communication protocol may be used, such as, for example, a master/slave communication protocol, server/client communication protocol, peer/peer communication protocol, or any combination thereof. For example, using a master/slave communication protocol, one of the devices, the master device, controls the other device, the slave device. For instance, the portable electronic device may become a slave to the head-mounted display EEG device such that the head-mounted display EEG device controls the operation of the portable electronic device once they are coupled. Alternatively, the head-mounted display EEG device can serve as a slave of the portable electronic device by simply implementing actions based on controls from the portable electronic device. As another example, using a client/server communication protocol, a server program, operating on either portable electronic device or head-mounted display EEG device, responds to requests from a client program. As yet another example, using a peer-to-peer communication protocol, either of the two devices can initiate a communication session.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

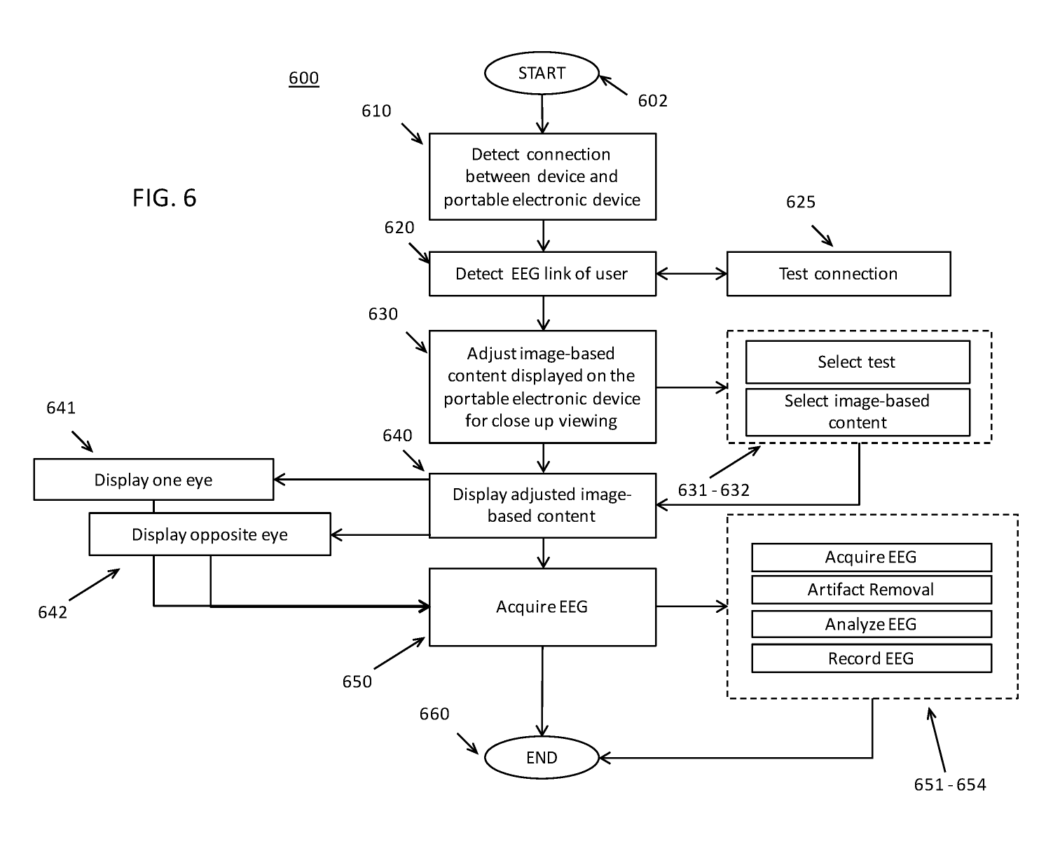

FIG. 6 shows a flowchart of an illustrative process 600 for displaying image-based content on a portable electronic device in accordance with one embodiment of the invention. In the discussion below, the head-mounted EEG display system includes a head-mounted display EEG device and a portable electronic device coupled to the device.

Process starts at step 602. At step 610, the head-mounted EEG display system can detect the connection between the head-mounted display EEG device and the portable electronic device. For example, the connection can either be wired or wireless. After a connection has been detected, process 600 moves to step 620. At step 620, the system can detect the connection of the EEG sensors by testing connection 625 with the user's head and require adjustment by the user. Once a robust connection between sensors and user has been detected, the head-mounted EEG display system can adjust image-based content displayed 630 on the portable electronic device for close up viewing. After the image-based content has been adjusted, process 600 moves to step 640, or if multiple tests are available, user can select the test 631 and the corresponding image based content 632 to present. At step 640, the head-mounted EEG display system can display the adjusted image-based content (e.g., visual stimulus) to the user. For example, a display screen on the portable electronic device can project the adjusted image-based content to the user. Display can occur on both eyes or separately 641 and 642. Process 600 then moves to step 650, wherein the system acquires EEG signal which correlates to the evoked potentials of the visual stimulus. Process 600 then stops at step 660. An exemplary system can employ dry microelectromechanical system EEG sensors, low-power signal acquisition, amplification and digitization, wireless telemetry, online artifact cancellation and real-time processing. In addition, the present technology can include analytical techniques, including machine learning or signal separation techniques 651-654 such as principal component analysis or independent component analysis, which can improve detectability of VERP signals.

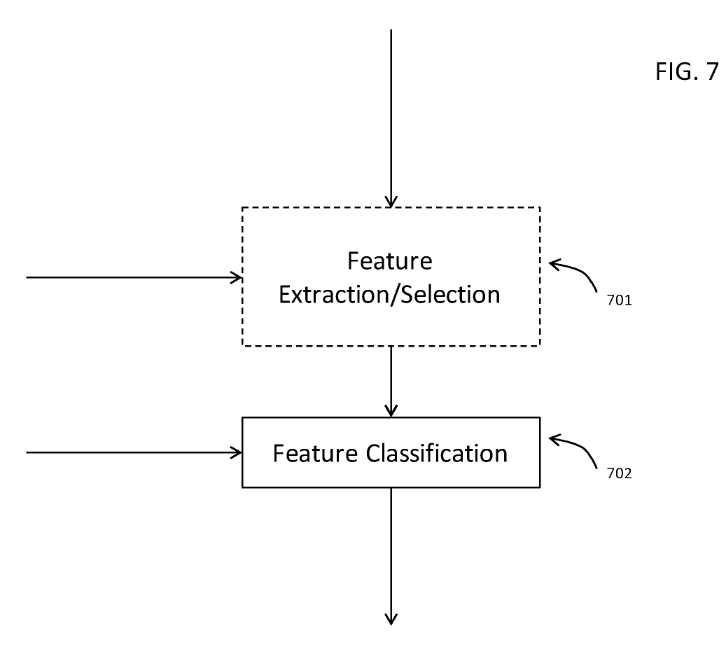

FIG. 7 illustrates an exemplary, non-limiting system that employs a learning component, which can facilitate automating one or more processes in accordance with the disclosed aspects. A memory (not illustrated), a processor (not illustrated), and a feature classification component 702, as well as other components (not illustrated) can include functionality, as more fully described herein, for example, with regard to the previous figures. A feature extraction component 701, and/or a feature selection component 701, of reducing the number of random variables under consideration can be utilized, although not necessarily, before performing any data classification and clustering. The objective of feature extraction is transforming the input data into the set of features of fewer dimensions. The objective of feature selection is to extract a subset of features to improve computational efficiency by removing redundant features and maintaining the informative features.

Classifier 702 may implement any suitable machine learning or classification technique. In one embodiment, classification models can be formed using any suitable statistical classification or machine learning method that attempts to segregate bodies of data into classes based on objective parameters present in the data. Machine learning algorithms can be organized into a taxonomy based on the desired outcome of the algorithm or the type of input available during training of the machine. Supervised learning algorithms are trained on labeled examples, i.e., input where the desired output is known. The supervised learning algorithm attempts to generalize a function or mapping from inputs to outputs which can then be used speculatively to generate an output for previously unseen inputs. Unsupervised learning algorithms operate on unlabeled examples, i.e., input where the desired output is unknown. Here the objective is to discover structure in the data (e.g. through a cluster analysis), not to generalize a mapping from inputs to outputs. Semi-supervised learning combines both labeled and unlabeled examples to generate an appropriate function or classifier. Transduction, or transductive inference, tries to predict new outputs on specific and fixed (test) cases from observed, specific (training) cases. Reinforcement learning is concerned with how intelligent agents ought to act in an environment to maximize some notion of reward. The agent executes actions that cause the observable state of the environment to change. Through a sequence of actions, the agent attempts to gather knowledge about how the environment responds to its actions, and attempts to synthesize a sequence of actions that maximizes a cumulative reward. Learning to learn learns its own inductive bias based on previous experience. Developmental learning, elaborated for robot learning, generates its own sequences (also called curriculum) of learning situations to cumulatively acquire repertoires of novel skills through autonomous self-exploration and social interaction with human teachers, and using guidance mechanisms such as active learning, maturation, motor synergies, and imitation. Machine learning algorithms can also be grouped into generative models and discriminative models.

In one embodiment of the present invention, classification method is a supervised classification, wherein training data containing examples of known categories are presented to a learning mechanism, which learns one or more sets of relationships that define each of the known classes. New data may then be applied to the learning mechanism, which then classifies the new data using the learned relationships. In supervised learning approaches, the controller or converter of neural impulses to the device needs a detailed copy of the desired response to compute a low-level feedback for adaptation.

Examples of supervised classification processes include linear regression processes (e.g., multiple linear regression (MLR), partial least squares (PLS) regression and principal components regression (PCR)), binary decision trees (e.g., recursive partitioning processes such as CART), artificial neural networks such as back propagation networks, discriminant analyses (e.g., Bayesian classifier or Fischer analysis), logistic classifiers, and support vector classifiers (support vector machines). Another supervised classification method is a recursive partitioning process.

Additional examples of supervised learning algorithms include averaged one-dependence estimators (AODE), artificial neural network (e.g., backpropagation, autoencoders, Hopfield networks, Boltzmann machines and Restricted Boltzmann Machines, spiking neural networks), Bayesian statistics (e.g., Bayesian classifier), case-based reasoning, decision trees, inductive logic programming, gaussian process regression, gene expression programming, group method of data handling (GMDH), learning automata, learning vector quantization, logistic model tree, minimum message length (decision trees, decision graphs, etc.), lazy learning, instance-based learning (e.g., nearest neighbor algorithm, analogical modeling), probably approximately correct learning (PAC) learning, ripple down rules, a knowledge acquisition methodology, symbolic machine learning algorithms, support vector machines, random forests, decision trees ensembles (e.g., bagging, boosting), ordinal classification, information fuzzy networks (IFN), conditional random field, ANOVA, linear classifiers (e.g., Fisher's linear discriminant, logistic regression, multinomial logistic regression, naive Bayes classifier, perceptron), Quadratic classifiers, k-nearest neighbor, decision trees, and Hidden Markov models.

In other embodiments, the classification models that are created can be formed using unsupervised learning methods. Unsupervised learning is an alternative that uses a data driven approach that is suitable for neural decoding without any need for an external teaching signal. Unsupervised classification can attempt to learn classifications based on similarities in the training data set, without pre-classifying the spectra from which the training data set was derived.

Approaches to unsupervised learning include:
clustering (e.g., k-means, mixture models, hierarchical clustering), (Hastie, Trevor, Robert Tibshirani, Friedman, Jerome (2009). The Elements of Statistical Learning: Data mining, Inference, and Prediction. New York: Springer. pp. 485-586)
hidden Markov models,
blind signal separation using feature extraction techniques for dimensionality reduction (e.g., principal component analysis, independent component analysis, non-negative matrix factorization, singular value decomposition) (Acharyya, Ranjan (2008); A New Approach for Blind Source Separation of Convolutive Sources, ISBN 978-3-639-07797-1 (this book focuses on unsupervised learning with Blind Source Separation))

Among neural network models, the self-organizing map (SOM) and adaptive resonance theory (ART) are commonly used unsupervised learning algorithms. The SOM is a topographic organization in which nearby locations in the map represent inputs with similar properties. The ART model allows the number of clusters to vary with problem size and lets the user control the degree of similarity between members of the same clusters by means of a user-defined constant called the vigilance parameter. ART networks are also used for many pattern recognition tasks, such as automatic target recognition and seismic signal processing. The first version of ART was “ART1”, developed by Carpenter and Grossberg (1988) (Carpenter, G. A. and Grossberg, S. (1988). “The ART of adaptive pattern recognition by a self-organizing neural network”. Computer 21: 77-88).

In one embodiment, a support vector machine (SVM) is an example of a classifier that can be employed. The SVM can operate by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches include, for example, naïve Bayes, Bayesian networks, decision trees, neural networks, fuzzy logic models, and probabilistic classification models providing different patterns of independence can be employed. Classification as used herein also may be inclusive of statistical regression that is utilized to develop models of priority.

The disclosed aspects can employ classifiers that are explicitly trained (e.g., via user intervention or feedback, preconditioned stimuli such as known EEG signals based on previous stimulation, and the like) as well as implicitly trained (e.g., via observing VERP, observing patterns, receiving extrinsic information, and so on), or combinations thereof. For example, SVMs can be configured via a learning or training phase within a feature classifier constructor and feature selection module. Thus, the classifier(s) can be used to automatically learn and perform a number of functions, including but not limited to learning bio-signals for particular VERPs, removing noise including artifact noise, and so forth. The learning can be based on a group or specific for the individual. The criteria can include, but is not limited to, EEG fidelity, noise artifacts, environment of the device, application of the device, preexisting information available, and so on.

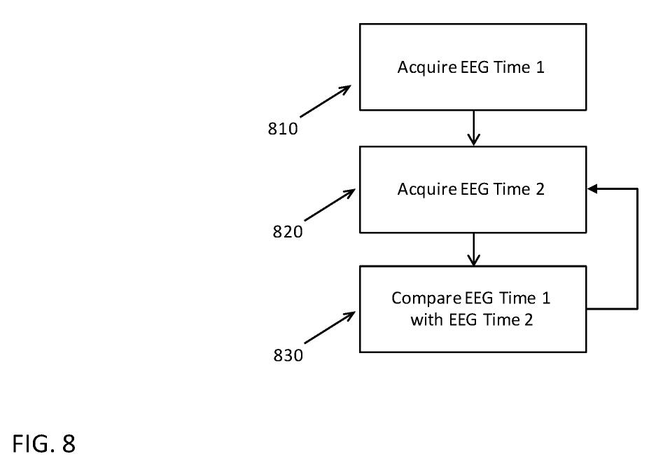

FIG. 8 illustrates a process 800 for comparing the acquired/analyzed EEG signals from the user over time. A disparity of signals acquired over time can indicate potential complications and/or degeneration of neurons or other cells. A measurement is made at first time point 810, which can be used as the control or reference. A measurement is then made at a second time point 820 which may be at any time period after the first time point, e.g., second(s), hour(s), day(s), week(s), month(s), year(s), etc. The signal of the first time point is compared 830 with the signal of the second time point. The signal can refer to the EEG signal or parameters surrounding the EEG signal, such as the delay in acquiring the EEG after visual stimulation, etc. Measurements can be repeated and comparisons made in the aggregate or individually.

In one embodiment, the present invention further comprises a neurofeedback loop. Neurofeedback is direct training of brain function, by which the brain learns to function more efficiently. We observe the brain in action from moment to moment. We show that information back to the person. And we reward the brain for changing its own activity to more appropriate patterns. This is a gradual learning process. It applies to any aspect of brain function that we can measure. Neurofeedback is also called EEG Biofeedback, because it is based on electrical brain activity, the electroencephalogram, or EEG. Neurofeedback is training in self-regulation. It is simply biofeedback applied to the brain directly. Self-regulation is a necessary part of good brain function. Self-regulation training allows the system (the central nervous system) to function better. Neurofeedback is a type of biofeedback that measures brain waves to produce a signal that can be used as feedback to teach self-regulation of brain function. Neurofeedback is commonly provided using video or sound, with positive feedback for desired brain activity and negative feedback for brain activity that is undesirable.

Neurofeedback addresses problems of brain disregulation. These happen to be numerous. They include the anxiety-depression spectrum, attention deficits, behavior disorders, various sleep disorders, headaches and migraines, PMS and emotional disturbances. It is also useful for organic brain conditions such as seizures, the autism spectrum, and cerebral palsy.

Thus it is seen that systems and methods are provided for allowing users to couple a portable electronic device in the head-mounted display EEG device. It is also seen that systems and methods are provided for allowing users to see the outside world while wearing a head-mounted display EEG device. Persons skilled in the art will appreciate that the invention can be practiced by other than the described embodiments, which are presented for purposes of illustration and not of limitation, and the present invention is limited only by the claims which follow.

1. A head mounted neuro-monitoring device for monitoring electrical brain activity associated with visual field of a user comprising
a. a sensor unit to acquire electroencephalogram (EEG) signals from one or more electroencephalograph (EEG) sensors arranged to acquire EEG signals from the head of a user and
b. a portable electronic device frame capable of housing a removable portable electronic device with a visual display unit that is positioned in front of the user's eyes to present visual stimuli, in which the visual stimuli is configured to evoke visual-event-related responses (VERPs) in the EEG signals exhibited by the user and acquired by the sensor unit.

2. The device of claim 1, wherein the one or more electrodes is a ground or reference terminal.

3. The device of claim 2, wherein the electrodes are replaceable.

4. The device of claim 1, wherein the device comprises two or more electrodes arranged in an array to circumnavigate headband to record EEG signals across the parieto-occipital region of the brain.

5. The device of claim 1, further comprising a data processing unit to process multiple EEG signals and communicate with the sensor unit and the portable electronic device

6. The device of claim 5, wherein the processing unit is capable of improving the quality of the EEG signals, estimating the parameter values and/or classifying the characteristics of the biological signals captured by the sensors.

7. The device of claim 1, wherein the portable electronic device is selected from a smartphone or tablet device.

8. The device of claim 1 further comprising an adjustable optical interface mechanism that provides focused proximal viewing of an image on the portable electronic device.

9. The device of claim 1, further comprising a means for monitoring movements of the user's eyes to determine instances associated with the user gazing away from the center of the visual field.

10. The device of claim 9, further comprising an electrooculogram (EOG) unit including one or more electrodes to be placed proximate the outer canthus of each of the user's eyes to measure corneo-retinal standing potential (CRSP) signals, wherein the one or more electrodes of the EOG unit are in communication with the data processing unit to process the acquired CRSP signals from the one or more electrodes to determine movements of the user's eyes.

11. The device of claim 10, further comprising an electromyogram (EMG), electrocardiography (ECG), and/or electrodermal activity (EDA);

12. The device of claim 9, further comprising an eye tracking device including a camera employed in the wearable visual display unit and in communication with the data processing unit, wherein the camera is operable to record images of the user's eyes.

13. A neuro-monitoring system for monitoring electrical brain activity associated with visual field of a user comprising a head mounted neuro-monitoring device, wherein device comprises:
a. a sensor unit to acquire electroencephalogram (EEG) signals from one or more electroencephalograph (EEG) sensors arranged to acquire EEG signals from the head of a user,
b. a portable electronic device frame capable of housing a removable portable electronic device in a position in front of the user's eyes, and
c. a portable electronic device with a display screen to present visual stimuli to the user in a plurality of sectors of a visual field, in which the visual stimuli is configured to evoke visual-event-related responses (VERPs) in the EEG signals exhibited by the user and acquired by the sensor unit.

14. The system of claim 13, wherein the portable electronic device is configured to present visual stimulus to diagnose neurological complications.

15. The system of claim 13, wherein the complications are ocular pathologies degenerative diseases, or other mental disorders.

16. The system of claim 13, wherein the system is configured for business and marketing applications, educational and learning applications or for entertainment purposes.

17. The system of claim 13, wherein the system further comprises a remote server to interface with the device.

18. The system of claim 13, wherein the system further comprises a neurofeedback loop to train the user to exhibit desired brain function.

19. A method for monitoring, tracking, and/or diagnosing various paradigms in cognitive function and clinical neuroscience that produce detectable and distinguishable responses to visual stimulation or visual event cues, comprising:
presenting to a user visual stimuli in a plurality of sectors of a visual field of a subject from a portable head-mounted EEG display device, wherein for each sector the presented visual stimuli includes an optical flickering effect at a selected frequency;
acquiring electroencephalogram (EEG) signals from one or more electrodes in contact with the head of the subject;
processing the acquired EEG signals by improving the biological signal quality, estimating the parameter values and/or classifying the characteristics of user's event-related neural activities,
analyzing the parameter values and/or the classification of user's event-related neural activities to attain a visual-event-related potential (VERP), and
producing a quantitative assessment of the visual field of the subject based on the VERP data.

20. The method of claim 19, comprising monitoring changes to the VERP over time.
